(19) United States
(12) Patent Application Publication
(10) Pub. No.: US 2017/0254805 A1
(43) Pub. Date: Sep. 7, 2017

(54) BIOCHEMICAL MOLECULE DETECTION SENSOR AND METHOD FOR DETECTING SPECIFIC MOLECULE USING MULTI-WAVELENGTH FLUORESCENCE

(71) Applicant: RESEARCH & BUSINESS FOUNDATION SUNGKYUNKWAN UNIVERSITY, Suwon-si (KR)

(72) Inventors: Nae Eung LEE, Seoul (KR); Sajal SHRIVASTAVA, Suwon-si (KR); Young Min SON, Suwon-si (KR); Won Il LEE, Suwon-si (KR)

(73) Assignee: RESEARCH BUSINESS FOUNDATION SUNGKYUNKWAN UNIVERSITY, Suwon-si (KR)

(21) Appl. No.: 15/449,049

(22) Filed: Mar. 3, 2017

(30) Foreign Application Priority Data
Mar. 3, 2016 (KR) 10-2016-0025464
Feb. 2, 2017 (KR) 10-2017-0014843

(51) Int. Cl. G01N 33/543

(52) U.S. Cl. CPC G01N 33/54386; G01N 33/54353

(57) ABSTRACT

A biochemical molecule detection sensor includes a substrate; a first aptamer complex chemically bound to the substrate, wherein the first aptamer complex selectively binds to a biochemical molecule, and includes at least one double helix DNA into which a fluorescent dye generating first fluorescence with a first wavelength is intercalated; and at least one second aptamer complex selectively binding to the biochemical molecule and generating second fluorescence with a second wavelength, wherein the first wavelength is different from the second wavelength, wherein fluorescence intensities of the first and second fluorescence depend on an amount of the biochemical molecule reacting with the first aptamer complex and the second aptamer complex.

### CROSS-REFERENCE TO RELATED APPLICATION

This application claims the benefit of Korean Patent Application Nos. 10-2016-0025464 filed Mar. 3, 2016 and 10-2017-0014843 filed Feb. 2, 2017, the entire contents of each of which are incorporated herein by reference.

### BACKGROUND

Field of the Invention

The present disclosure relates to a biochemical molecule detection sensor to detect a biochemical molecule and to quantify an unknown concentration of a biochemical molecule. More particularly, the present disclosure relates to a biochemical molecule detection sensor to quantify an unknown concentration of a biochemical molecule and to improve an accuracy in detection of a biochemical molecule, and further relates to a method for detecting a biochemical molecule using multi-wavelength fluorescence.

Discussion of Related Art

Conventional enzyme-linked immunosorbent assays or enzyme-linked aptamer assays have been used as standard approach for biomolecular detection using fluorescence. The conventional fluorescence detection technology may involve labeling a bio-receptor with a fluorescent substance of a single wavelength, adding a sample containing a target biomolecule into the bio-receptor labelled with the fluorescent substance, and measuring fluorescence intensity for the single wavelength. However, error-causes such as a background noise of wavelengths similar to those of the fluorescent substance or other molecules other than a target biomolecule non-specifically bound to the bio-receptor may lower the detection accuracy.

In addition, although, in the prior art, thorough washing is performed at each of various steps of binding a bio-receptor having a fluorescent substance labelled therewith to a target biomolecule, some residual intermediate bio-receptors cause detection result errors. Further, the detection accuracy is further lowered due to the background noise of the similar wavelength band to those of the labelled fluorescent substance.

In order to solve such problems, there have been made reports on detection methods using direct interaction between target biomolecules and bio-receptors. However, they have also been unable to reduce the errors due to nonspecific binding and background noise in a single platform.

### SUMMARY OF THE INVENTION

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

The present disclosure is to provide a biochemical molecule detection sensor to quantify an unknown concentration of a biochemical molecule and to improve an accuracy in detection of a biochemical molecule, and further relates to a method for detecting a biochemical molecule using multi-wavelength fluorescence.

In one aspect of the present disclosure, there is provided a biochemical molecule detection sensor comprising: a substrate; a first aptamer complex chemically bound to the substrate, wherein the first aptamer complex selectively binds to a biochemical molecule, and includes at least one double helix DNA into which a fluorescent dye generating first fluorescence with a first wavelength is intercalated; and at least one second aptamer complex selectively binding to the biochemical molecule and generating second fluorescence with a second wavelength, wherein the first wavelength is different from the second wavelength, wherein fluorescence intensities of the first and second fluorescence depend on an amount of the biochemical molecule reacting with the first aptamer complex and the second aptamer complex.

In one implementation of the above-defined sensor, the double helix DNA includes: a first nucleic acid strand having a first nucleotide sequence selectively binding to the biochemical molecule; and a second nucleic acid strand having a second nucleotide sequence complementary to the first nucleotide sequence, wherein the second nucleic acid strand is complementary to the first nucleic acid strand, wherein the fluorescent dye is intercalated into between the first nucleic acid strand and the second nucleic acid strand.

In one implementation of the above-defined sensor, the fluorescent dye includes at least one selected from a group consisting of SYBR Green I, thiazole orange, and ethidium bromide.

In one implementation of the above-defined sensor, the second aptamer complex includes: a third nucleic acid strand having the first nucleotide sequence selectively binding to the biochemical molecule; and a phosphor coupled to the third nucleic acid strand, wherein the phosphor generates the second fluorescence.

In one implementation of the above-defined sensor, the first nucleic acid strand is labeled with a functional group chemically bound to the substrate, wherein the functional group includes at least one selected from a group consisting of amine group, carboxyl group, maleimide group or thiol group.

In one implementation of the above-defined sensor, the phosphor includes at least one selected from a group consisting of quantum dot, fluorescent dye and metal nanocluster.

In one aspect of the present disclosure, there is provided a method for detecting a biochemical molecule using multi-wavelength fluorescence, the method comprising: (a) applying a reagent onto a first aptamer complex, wherein the first aptamer complex is chemically bound to the substrate, wherein the first aptamer complex selectively binds to the biochemical molecule, and includes at least one double helix DNA into which a fluorescent dye generating first fluorescence with a first wavelength is intercalated, wherein the reagent includes the biochemical molecule and at least one second aptamer complex selectively binding to the biochemical molecule and generating second fluorescence with a second wavelength, wherein the first wavelength is different from the second wavelength; (b) removing the second aptamer complex not bound to the biochemical molecule; and (c) measuring fluorescence intensities of the first and second fluorescence.

In one implementation of the above-defined method, the double helix DNA includes: a first nucleic acid strand having a first nucleotide sequence selectively binding to the biochemical molecule; and a second nucleic acid strand having a second nucleotide sequence complementary to the first nucleotide sequence, wherein the second nucleic acid strand is complementary to the first nucleic acid strand, wherein the fluorescent dye is intercalated into between the first nucleic acid strand and the second nucleic acid strand.

In one implementation of the above-defined method, the second aptamer complex includes: a third nucleic acid strand having the first nucleotide sequence selectively binding to the biochemical molecule; and a phosphor coupled to the third nucleic acid strand, wherein the phosphor generates the second fluorescence.

In one implementation of the above-defined method, the method further comprises repeating the operations (a) to (c) using varying concentrations of the biochemical molecule; and deriving an analysis function for fluorescence intensities of the first and second fluorescence based on the concentration of the biochemical molecule.

In one implementation of the above-defined method, the method further comprises detecting the biochemical molecule by determining whether the intensity of the first fluorescence increases or decreases and whether the second fluorescence occurs or not.

In one implementation of the above-defined method, the method further comprises detecting the biochemical molecule by determining whether the intensity of the first fluorescence increases or decreases and whether the intensity of the second fluorescence increases or decreases.

In one implementation of the above-defined method, the operation (c) is carried out by imaging using a camera.

The present sensor and method can quantify an unknown concentration of a biochemical molecule. Further, the present sensor and method can improve the accuracy and reliability of detection results of specific biochemical molecules by detecting biochemical molecules using fluorescence of two wavelengths.

### BRIEF DESCRIPTION OF THE DRAWINGS

The accompanying drawings, which are incorporated in and form a part of this specification and in which like numerals depict like elements, illustrate embodiments of the present disclosure and, together with the description, serve to explain the principles of the disclosure.

FIG. 1 is a conceptual diagram of a biochemical molecule detection sensor according to an embodiment of the present disclosure.

FIG. 2 is a conceptual diagram for explaining a reaction state after a biochemical molecule and a second aptamer complex are introduced into a biochemical molecule detection sensor according to an embodiment of the present disclosure.

FIG. 3 is a graph showing a fluorescence intensity of a fluorescent dye based on a concentration of a biochemical molecule.

FIG. 4 is a graph showing a fluorescence intensity of a phosphor based on a concentration of a biochemical molecule.

FIG. 5 is a graph showing a fluorescence intensity of a fluorescent dye and phosphor with respect to wavelengths.

FIG. 6 is a flowchart of a method for detecting a biochemical molecule using multi-wavelength fluorescence according to an embodiment of the present disclosure.

FIG. 7 is a flowchart of a method for detecting a biochemical molecule using multi-wavelength fluorescence according to another embodiment of the present disclosure.

FIG. 8A is a graph showing a concentration of ATP and a fluorescence intensity change of SYBR Green I based on a concentration of a drug (5-FU) injected to cells producing ATP.

FIG. 8B is a graph showing a concentration of ATP and a fluorescence intensity change of silver nanoclusters based on a concentration of a drug (5-FU) injected to cells producing ATP.

FIG. 8C is a graph showing a ratio of a fluorescence intensity ratio or change of SYBR Green I to a fluorescence intensity ratio or change of silver nanoclusters based on a concentration of a drug (5-FU) injected to cells producing ATP.

FIG. 9A is a graph showing a concentration of ATP and a fluorescence intensity change of SYBR Green I based on concentration of a drug (Gemcitabine) injected into ATP-producing cells.

FIG. 9B is a graph showing a concentration of ATP and a fluorescence intensity change of silver nanoclusters based on a concentration of a drug (Gemcitabine) injected into cells producing ATP.

FIG. 9C is a graph showing a ratio of a fluorescence intensity ratio or change of SYBR Green I to a fluorescence intensity ratio or change of silver nanoclusters based on a concentration of a drug (Gemcitabine) injected into cells producing ATP.

FIG. 10 shows quantification data based on concentrations of a target biomolecule for two fluorescence wavelength bands, achieved using a smartphone camera and a biochemical molecule detection sensor according to the present disclosure.

### DETAILED DESCRIPTION OF THE INVENTION

Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

It will be understood that when an element or layer is referred to as being “connected to”, or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of the associated listed items. Expression such as “at least one of” when preceding a list of elements may modify the entire list of elements and may not modify the individual elements of the list.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the following description, numerous specific details are set forth in order to provide a thorough understanding of the present disclosure. The present disclosure may be practiced without some or all of these specific details. In other instances, well-known process structures and/or processes have not been described in detail in order not to unnecessarily obscure the present disclosure.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.”

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

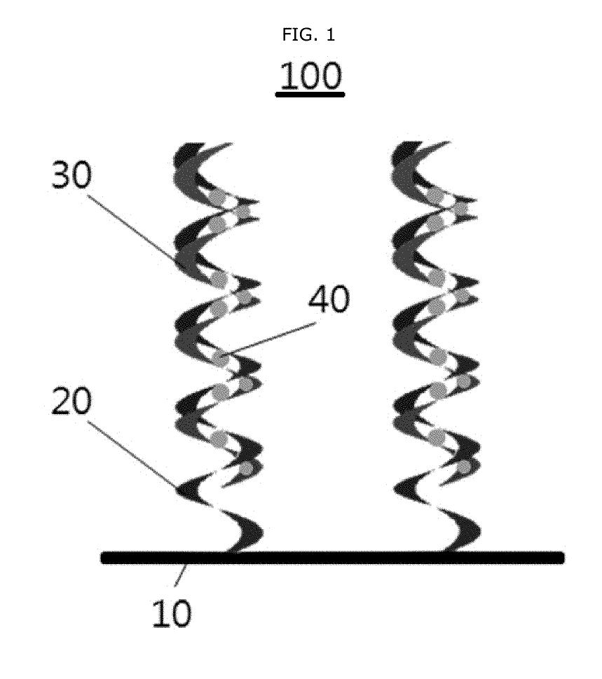

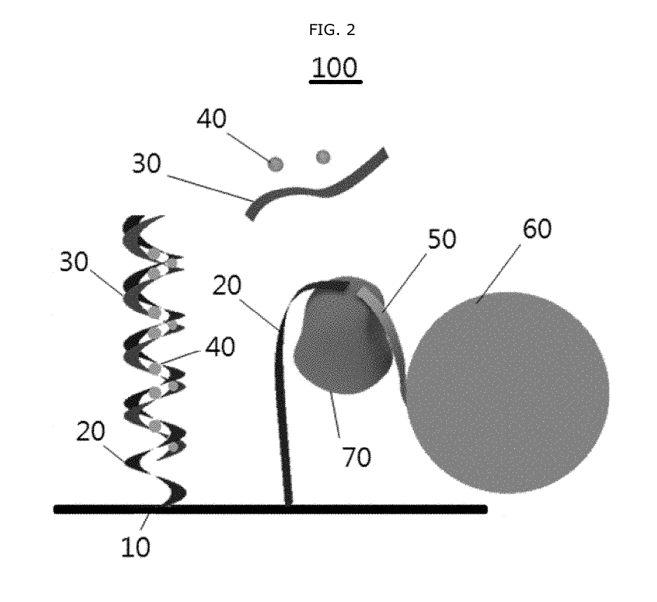

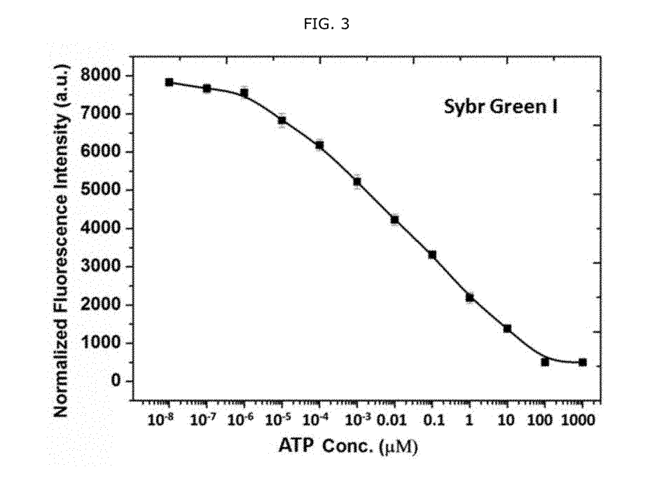

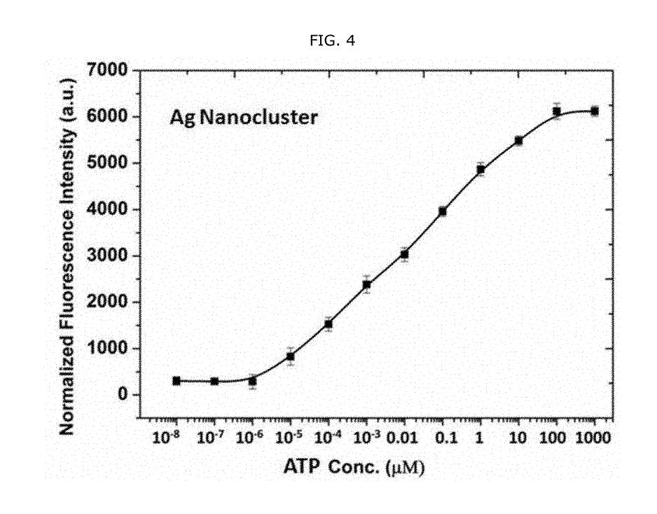

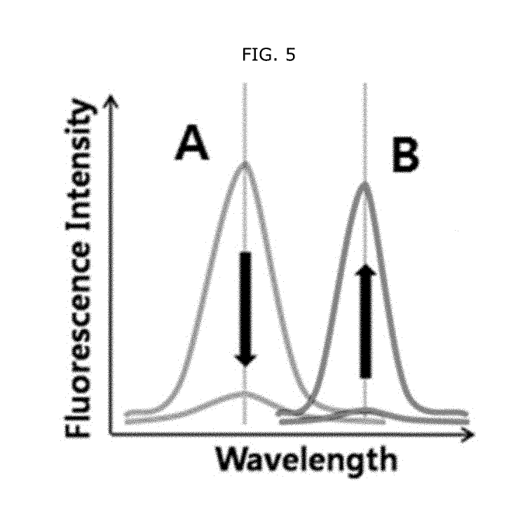

FIG. 1 is a conceptual diagram of a biochemical molecule detection sensor according to an embodiment of the present disclosure. FIG. 2 is a conceptual diagram for explaining a reaction state after a biochemical molecule and a second aptamer complex are introduced into a biochemical molecule detection sensor according to an embodiment of the present disclosure. FIG. 3 is a graph showing a fluorescence intensity of a fluorescent dye based on a concentration of a biochemical molecule. FIG. 4 is a graph showing a fluorescence intensity of a phosphor based on a concentration of a biochemical molecule. FIG. 5 is a graph showing a fluorescence intensity of a fluorescent dye and phosphor with respect to wavelengths.

Referring to FIG. 1, a biochemical molecule detection sensor 100 according to an embodiment of the present disclosure may include a substrate 10, a first aptamer complex, and a second aptamer complex, each complex including at least one double helix DNA.

The substrate 10 may be made of glass, polymer or paper. Further, the surface of the substrate 10 may be functionalized. As an example, the surface of the substrate 10 can be functionalized using 2 wt % 3-aminopropyltriethoxysilane (APTES) solution, and, in turn, 2 wt % glutaraldehyde solution. The functionalization of the surface of substrate 10 is intended to allow chemical bonding between the substrate 10 and the double helix DNA of the first aptamer complex. The double helix DNA is immobilized on the substrate 10 and can be selectively bound to the biochemical molecule 70.

At least one first aptamer complex may be chemically bound to the substrate 10 and may selectively bind to a biochemical molecule. To this end, the first aptamer complex may contain double helix DNA. The double helix DNA may contain a first nucleic acid strand 20 and a second nucleic acid strand 30. A fluorescent dye 40 may be intercalated into the double helix DNA, wherein the dye 40 generates a first wavelength of fluorescence. The first nucleic acid strand 20 may have a first nucleotide sequence that can selectively bind to the biochemical molecule 70. The second nucleic acid strand 30 may have a second nucleotide sequence complementary to the first nucleotide sequence and thus may act as a complementary strand to the first nucleic acid strand 20. The biochemical molecule 70 may be an ATP molecule. However, the present disclosure is not limited thereto. As the biochemical molecule 70 varies, the first nucleotide sequence may vary so as to be bound to the varied biochemical molecule.

The first nucleic acid strand 20 may be labeled with a functional group, for example, an amine group, a carboxyl group, a maleimide group, or a thiol group. This labelling of the functional group may allow the chemical bonding between the functionalized substrate 10 and the first nucleic acid strand 20.

The first nucleic acid strand 20 and second nucleic acid strand 30 may be hybridized to form the double helix DNA. In this connection, the first nucleic acid strand 20 and the second nucleic acid strand 30 with the same molar concentration (for example, 1 uM) may be mixed to form a mixture which in turn may be double-boiled at about 93° C. for about 3 minutes, and, thereafter, may be cooled at room temperature for about 50 minutes. In this way, the first nucleic acid strand 20 and the second nucleic acid strand 30 may be hybridized to form the double helix DNA 20 and 30.

The fluorescent dye 40 may generate a first wavelength of fluorescence. The fluorescent dye 40 may be intercalated into between the first nucleic acid strand 20 and second nucleic acid strand 30. In one example, the fluorescent dye 40 may be intercalated into double helix DNA by exposing the double helix DNA to the fluorescent dye 40. Example of the fluorescent dye 40 may include, but be limited to, one or more of SYBR Green I (SGI), thiazole orange and ethidium bromide. When the fluorescent dye 40 is the SYBR Green I, the first wavelength may be about 520 nm. The fluorescent dye 40 may produces fluorescence when intercalated into the double helix DNA, while the fluorescent dye 40 may not produce fluorescence when the fluorescent dye 40 is separated from the double helix DNA.

When the biochemical molecule 70 is bound to the double helix DNA, the fluorescent dye 40 may be separated from the double helix DNA to reduce the intensity of fluorescence generated in the double helix DNA. This is because when the fluorescent dye 40, which was intercalated into the double helix DNA, is separated therefrom, an amount of the fluorescent dye 40 in the double helix DNA reduces. When the decrease in the fluorescence intensity generated from the fluorescent dye 40 is confirmed, it may be determined that the biochemical molecule has been detected.

The at least one second aptamer complex may selectively bind to a biochemical molecule and may generate fluorescence of a second wavelength different from the first wavelength. The second aptamer complex may contain a third nucleic acid strand 50 and phosphor 60.

Referring to FIG. 2, the third nucleic acid strand 50 may have the first nucleotide sequence capable of selectively binding to the biochemical molecule 70.

The phosphor 60 has been coupled to the third nucleic acid strand 50. The phosphor 60 may generate a second wavelength of fluorescence that is different from the first wavelength of fluorescence that occurs in the fluorescent dye 40. For example, the phosphor 60 may include one or more of a quantum dot, a fluorescent dye and a metal nanocluster. The present disclosure is not limited thereto. Any fluorescent material may be used as the phosphor 60. In one example, the metal nanocluster may be embodied as a silver nanocluster (Ag nanocluster). The wavelength of the fluorescence generated in the Ag nanocluster may be about 650 nm.

The first and second aptamer complexes may be loaded into the biochemical molecule detection sensor 100 along with the biochemical molecule 70.

When the first and second aptamer complexes are introduced into the biochemical molecule detection sensor 100 along with the biochemical molecule 70, the biochemical molecule 70 becomes bound to the first nucleic acid strand 20, and, thus, the second nucleic acid strand 30, which has been hybridized to the first nucleic acid strand 20 becomes separated therefrom. In addition, the biochemical molecule 70 may bind to the third nucleic acid strand 50, thereby to form a linkage structure between the first nucleic acid strand 20, the biochemical molecule 70, the third nucleic acid strand 50 and the phosphor 60. This reaction reduces the intensity of fluorescence generated from the fluorescent dye 40, and, at the same time, fluorescence generated from the phosphor 60 may be detected. Therefore, it may be determined that the biochemical molecule has been detected when the fluorescence intensity from the fluorescent dye 40 is decreased and the fluorescence from phosphor 60 is detected.

The concentrations of the biochemical molecule 70 and complexes injected to the biochemical molecule detection sensor 100 may be increased. As the concentrations of the injected biochemical molecule 70 and the complexes are increased, the fluorescence intensity generated from the double helix DNA is reduced while the fluorescence intensity generated from the phosphor 60 is increased.

Referring to FIG. 3 and FIG. 4, as the concentration of the biochemical molecule increases, the fluorescence intensity of the fluorescent dye (SYBR Green I) decreases and the fluorescence intensity of the phosphor (Ag nanocluster) increases. Referring to FIG. 5, as the concentration of the biochemical molecule increases, the fluorescence intensity of the fluorescent dye (SYBR Green I, A) whose fluorescence wavelength is about 520 nm is reduced, while the fluorescence intensity of the phosphor (Ag nanocluster, B) whose fluorescence wavelength is about 650 nm is increased.

The biochemical molecule detection sensor 100 in accordance with one embodiment of the present disclosure can detect a biochemical molecule using fluorescence of two wavelengths. According to the present disclosure, a biochemical molecule can be detected more precisely because two wavelengths of fluorescence are used, in comparison with the detection of a biochemical molecule using one fluorescence wavelength, as will be described below in details.

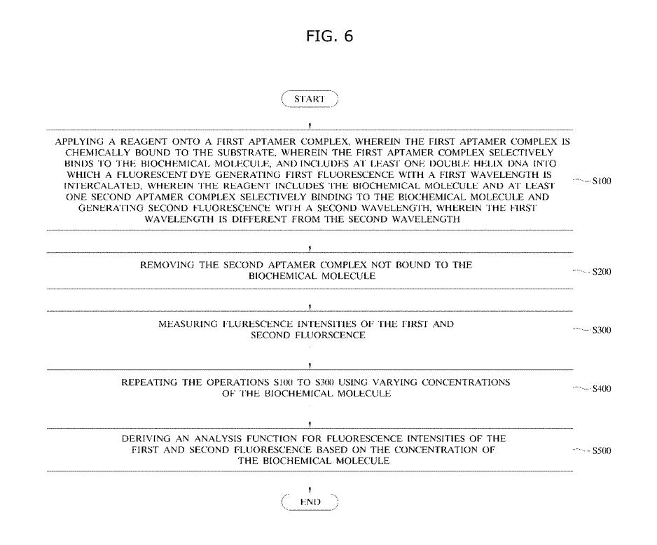

FIG. 6 is a flow chart of a biochemical molecule detection method using multi-wavelength fluorescence according to an embodiment of the present disclosure.

Referring to FIG. 6, the biochemical molecule detection method using a multi-wavelength fluorescence according to an embodiment of the present disclosure may include applying S100 a biochemical molecule and at least one second aptamer complex onto a first aptamer complex, wherein the first aptamer complex is chemically bound to the substrate, wherein the first aptamer complex selectively binds to the biochemical molecule, and includes at least one double helix DNA into which a fluorescent dye generating first fluorescence with a first wavelength is intercalated, wherein the at least one second aptamer complex selectively binds to the biochemical molecule and generates second fluorescence with a second wavelength, wherein the first wavelength is different from the second wavelength. The method may further include removing S200 the second aptamer complex not bound to the biochemical molecule, and measuring S300 fluorescence intensities of the first and second fluorescence. The method may further include repeating S400 the operations S100 to S300 using varying concentrations of the biochemical molecule; and deriving S500 an analysis function for fluorescence intensities of the first and second fluorescence based on the concentration of the biochemical molecule.

In order to detect the biochemical molecule using multi-wavelength fluorescence, in an operation S100 of the method, the biochemical molecule and the at least one second aptamer complex may be applied onto the first aptamer complex, wherein the first aptamer complex is chemically bound to the substrate, wherein the first aptamer complex selectively binds to the biochemical molecule, and includes at least one double helix DNA into which a fluorescent dye generating first fluorescence with a first wavelength is intercalated, wherein the at least one second aptamer complex selectively binds to the biochemical molecule and generates second fluorescence with a second wavelength, wherein the first wavelength is different from the second wavelength. In one example, a reagent including the biochemical molecule and second aptamer complex may be applied onto the first aptamer complex. In this connection, the application may mean that the first aptamer complex is exposed to the biochemical molecule and second aptamer complex, or the biochemical molecule and second aptamer complex are loaded onto the first aptamer complex.

When the biochemical molecule 70 and second aptamer complex are loaded on the first aptamer complex, the biochemical molecule 70 is bound to the first nucleic acid strand 20 of the biochemical molecule detection sensor 100, and thus the fluorescent dye 40 is separated from the double helix DNA of the biochemical molecule detection sensor 100. In this way, the fluorescence intensity of the first fluorescence can be reduced. In addition, when the biochemical molecule 70 is bound to the first nucleic acid strand 20, the phosphor 60 can be linked to the first nucleic acid strand 20 and thus the second fluorescence can be generated from the phosphor 60. The at least one second complex is preferably applied at a concentration such that the second complex sufficiently reacts with the biochemical molecule.

Upon completion of reaction between the first nucleic acid strand 20, biochemical molecule 70 and the complexes in the biochemical molecule detection sensor 100, the second aptamer complex unbound with the biochemical molecule is removed S200. The removal may be carried out through a cleaning process, which may use ionized water. Thus, the remaining unbound second aptamer complex may be removed.

After the second aptamer complex unbound to the biochemical molecule has been removed, measuring fluorescence intensities of the first and second fluorescence may be carried out S300.

The biochemical molecule detection method using multi-wavelength fluorescence according to one embodiment of the present disclosure may further include repeating S400 the operations S100 to S300 using varying concentrations of the biochemical molecule in order to derive a quantification analysis function based on a concentration of the biochemical molecule.

In this connection, first data on the first fluorescence intensity with the first wavelength generated from the first aptamer complex based on the concentration of the biochemical molecule and second data on the second fluorescence intensity with the second wavelength generated from the second aptamer complex based on the concentration of the biochemical molecule may be obtained. Using the first and second data, first and second quantification analysis functions can be derived.

Graphs according to the derived quantification analysis functions are shown in FIGS. 3 and 4, respectively. When using the quantification analysis functions, the concentration of the biochemical molecule with the unknown concentration can be determined by referencing the first and second quantification graphs. For example, an ATP molecule with an unknown concentration along with at least one second complex is injected into the biochemical molecule detection sensor 100, and, then, the measured first fluorescence intensity is applied to the graph according to the first quantification analysis function as shown in FIG. 3 in order to find out a corresponding ATP molecule concentration to the measured first fluorescence intensity. In this way, the exact concentration of the ATP molecule may be grasped. In the same manner, the measured second fluorescence intensity is applied to the graph according to the second quantification analysis function as shown in FIG. 4 in order to find out a corresponding ATP molecule concentration to the measured second fluorescence intensity. In this way, the exact concentration of the ATP molecule may be grasped. Thus, the present biochemical molecule detection method using the multi-wavelength fluorescence can accurately quantify biochemical molecules with unknown concentrations.

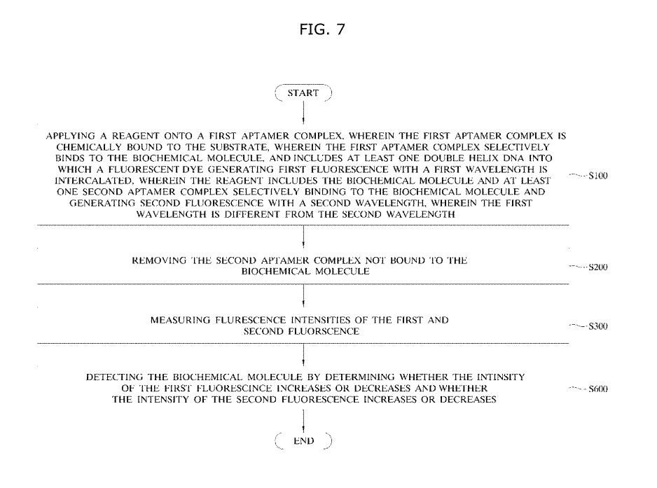

FIG. 7 is a flow chart of a biochemical molecule detection method using multi-wavelength fluorescence according to another embodiment of the present disclosure.

Referring to FIG. 7, the biochemical molecule detection method using a multi-wavelength fluorescence according to an embodiment of the present disclosure may include applying S100 a biochemical molecule and at least one second aptamer complex onto a first aptamer complex, wherein the first aptamer complex is chemically bound to the substrate, wherein the first aptamer complex selectively binds to the biochemical molecule, and includes at least one double helix DNA into which a fluorescent dye generating first fluorescence with a first wavelength is intercalated, wherein the at least one second aptamer complex selectively binds to the biochemical molecule and generates second fluorescence with a second wavelength, wherein the first wavelength is different from the second wavelength. The method may further include removing S200 the second aptamer complex not bound to the biochemical molecule, and measuring S300 fluorescence intensities of the first and second fluorescence. The method may further include detecting S600 the biochemical molecule by determining whether the intensity of the first fluorescence increases or decreases and whether the intensity of the second fluorescence increases or decreases. In this connection, whether the intensity of the second fluorescence increases or decreases may include whether the second fluorescence occurs or not. That is, the case when the intensity of the second fluorescence increases or decreases may include the case when the second fluorescence which disappears previously currently appears.

In order to detect the biochemical molecule using multi-wavelength fluorescence, in an operation S100 of the method, the biochemical molecule and the at least one second aptamer complex may be applied onto the first aptamer complex, wherein the first aptamer complex is chemically bound to the substrate, wherein the first aptamer complex selectively binds to the biochemical molecule, and includes at least one double helix DNA into which a fluorescent dye generating first fluorescence with a first wavelength is intercalated, wherein the at least one second aptamer complex selectively binds to the biochemical molecule and generates second fluorescence with a second wavelength, wherein the first wavelength is different from the second wavelength. In one example, a reagent including the biochemical molecule and second aptamer complex may be applied onto the first aptamer complex. In this connection, the application may mean that the first aptamer complex is exposed to the biochemical molecule and second aptamer complex, or the biochemical molecule and second aptamer complex are loaded onto the first aptamer complex.

When the biochemical molecule 70 and second aptamer complex are loaded on the first aptamer complex, the biochemical molecule 70 is bound to the first nucleic acid strand 20 of the biochemical molecule detection sensor 100, and thus the fluorescent dye 40 is separated from the double helix DNA of the biochemical molecule detection sensor 100. In this way, the fluorescence intensity of the first fluorescence can be reduced. In addition, when the biochemical molecule 70 is bound to the first nucleic acid strand 20, the phosphor 60 can be linked to the first nucleic acid strand 20 and thus the second fluorescence can be generated from the phosphor 60. The at least one second complex is preferably applied at a concentration such that the second complex sufficiently reacts with the biochemical molecule.

Upon completion of reaction between the first nucleic acid strand 20, biochemical molecule 70 and the complexes in the biochemical molecule detection sensor 100, the second aptamer complex unbound with the biochemical molecule is removed S200. The removal may be carried out through a cleaning process, which may use ionized water. Thus, the remaining unbound second aptamer complex may be removed.

After the second aptamer complex unbound to the biochemical molecule has been removed, measuring fluorescence intensities of the first and second fluorescence may be carried out S300.

When the biochemical molecule and at least one second aptamer complex are applied to the biochemical molecule detection sensor, the first fluorescence intensity decreases and the second fluorescence occurs or appears in a presence of the reaction between the biochemical molecule and the first and second aptamer complexes. To the contrary, when the biochemical molecule and at least one second aptamer complex are applied to the biochemical molecule detection sensor, the first fluorescence intensity remains constant and the second fluorescence does not occur or appear in an absence of the reaction between the biochemical molecule and the first and second aptamer complexes. However, even in the presence of the reaction between the biochemical molecule and the first and second aptamer complexes, the first fluorescence intensity may not decrease and the second fluorescence may not occur. In addition, even in the absence of the reaction between the biochemical molecule and the first and second aptamer complexes, the first fluorescence intensity may be decreased and the second fluorescence may occur. These errors may be caused by a variety of causes, such as some residual substances remaining even during the cleaning process, non-specific binding to a substance similar to the biochemical molecule, adsorption of the phosphor on the substrate, background noise of wavelength bands similar to those of the first fluorescence or second fluorescence, etc.

In order to solve these errors and to improve the accuracy of detection of biochemical molecules, the method for detection of the biochemical molecule by determining the increase or decrease of the first fluorescence intensity and the increase or decrease of the second fluorescence is as follows.

When the biochemical molecule and the at least one complexes are introduced into the biochemical molecule detection sensor, the following results may be measured. For convenience of explanation, the following explanation may be based on whether the first fluorescence intensity is increased or decreased and whether the second fluorescence occurs or not.

A first result in which the first fluorescence intensity decreases and the second fluorescence occurs: In this case, the biochemical molecule is detected. Thus, this case is defined as True Positive (TP). The TP may indicate that the biochemical molecule is normally detected.

A second result in which the first fluorescence intensity does not decrease and the second fluorescence does not occur: This case means that there is no biochemical molecule and, thus, this case is defined as True Negative (TN). The TN may indicate that the biochemical molecule is not normally detected.

A third result in which the first fluorescence intensity decreases but the second fluorescence does not occur: This is a case where the biochemical molecule detection sensor does not operate normally. Therefore, this case is defined as False Positive (FP).

A fourth result: the second fluorescence occurs without decreasing the first fluorescence intensity: This case is defined as False Negative (FN) because the biochemical molecule detection sensor does not operate normally. The above results are shown from Table 1 below.

TABLE 1

| | Second | |
|---|---|---|
| First fluorescence | fluorescence | |
| intensity decreases? | occurs? | Definition |
| Yes | Yes | True Positive (TP) |
| Yes | No | False Positive (FP) |
| No | Yes | False Negative (FN) |
| No | No | True Negative (TN) |

In general, when biochemical molecules are detected only by the detection using one fluorescence wavelength, FN may be determined incorrectly to be TN, and/or FP may be determined incorrectly to be TP. Thus, the biochemical molecule detection using the detection using one fluorescence wavelength may have lowered accuracy. In the present disclosure, the biochemical molecule detection method using multi-wavelength fluorescence may employ different fluorescence with two different wavelengths. Therefore, it is possible to grasp the error state accurately. Thus, the detection accuracy can be improved.

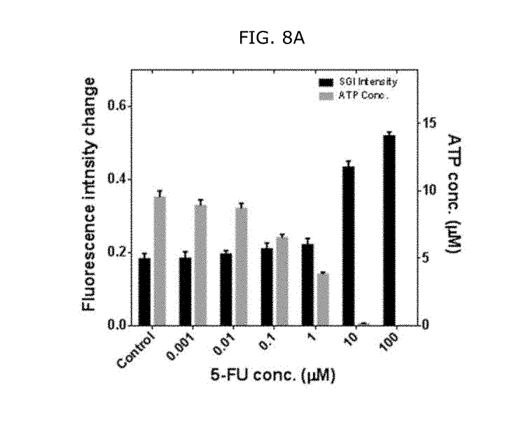

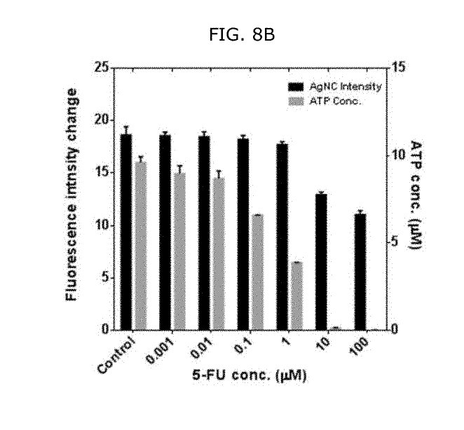

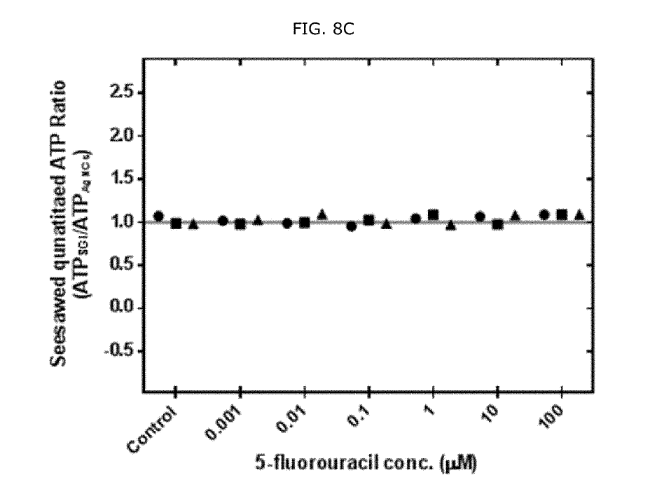

FIG. 8A is a graph showing a concentration of ATP and a fluorescence intensity change of SYBR Green I based on a concentration of a drug (5-FU) injected to cells producing ATP. FIG. 8B is a graph showing a concentration of ATP and a fluorescence intensity change of silver nanoclusters based on a concentration of a drug (5-FU) injected to cells producing ATP. FIG. 8C is a graph showing a ratio of a fluorescence intensity ratio or change of SYBR Green I to a fluorescence intensity ratio or change of silver nanoclusters based on a concentration of a drug (5-FU) injected to cells producing ATP.

Referring to FIG. 8A, it can be seen that as the concentration of a drug (5-FU) that can kill ATP-producing cells increases, the concentration of ATP produced in the cells decreases. As the concentration of ATP decreases, the fluorescence intensity change or ratio of SYBR Green I is increased. The fluorescence intensity change or ratio of SYBR Green I was measured as the ratio of the fluorescence intensity of SYBR Green I prior to the injection of the drug (5-FU) relative to the fluorescence intensity of SYBR Green I after the injection based on the concentration of the injected drug (5-FU).

Referring to FIG. 8B, it can be seen that as the concentration of the drug (5-FU) that can kill ATP-producing cells increases, the concentration of ATP decreases. As the concentration of ATP decreases, the fluorescence intensity ratio or change of silver nanocluster decreases. The fluorescence intensity ratio or change of the silver nanocluster was measured as the ratio of the fluorescence intensity of the silver nanocluster prior to the injection of the drug (5-FU) relative to the fluorescence intensity of the silver nanocluster after the injection based on the concentration of the injected drug (5-FU).

Referring to FIG. 8C, the ratio () of the fluorescence intensity ratio or change of SYBR Green I to the fluorescence intensity ratio or change of the silver nanocluster measured based on the concentration of drug 5-FU is close to 1. The change amount of fluorescence intensity of SYBR Green I corresponds to the change amount of fluorescence intensity of the Ag nanoclusters.

This indicates that as for the present biochemical molecule detection sensor, the biochemical molecule is normally bound to the complexes and the detection error is low, and, thus, the detection accuracy of the biochemical molecule is high.

This indicates that as for the present biochemical molecule detection sensor, the biochemical molecule is normally bound to the complexes and the detection error is low, and, thus, the detection accuracy of the biochemical molecule is high.

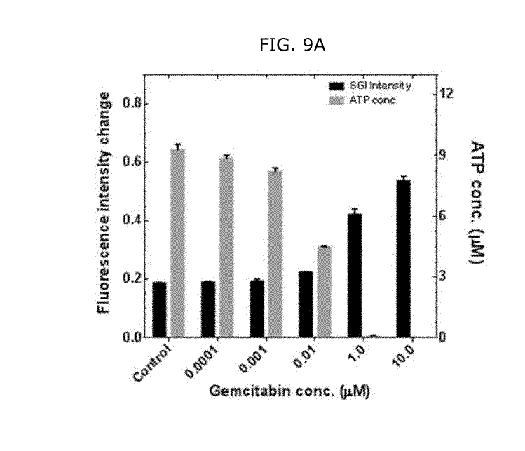

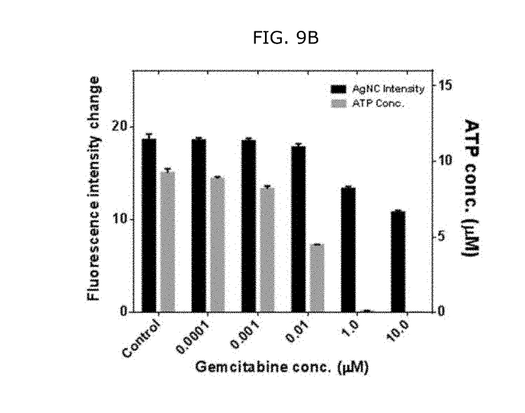

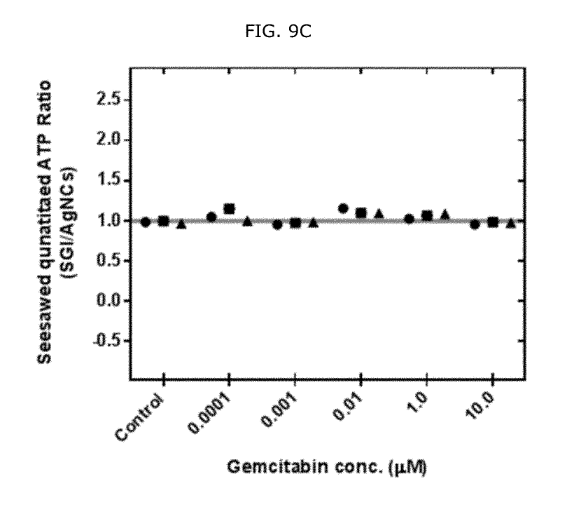

FIG. 9A is a graph showing a concentration of ATP and a fluorescence intensity change of SYBR Green I based on a concentration of a drug (Gemcitabine) injected to cells producing ATP. FIG. 9B is a graph showing a concentration of ATP and a fluorescence intensity change of silver nanoclusters based on a concentration of a drug (Gemcitabine) injected to cells producing ATP. FIG. 9C is a graph showing a ratio of a fluorescence intensity ratio or change of SYBR Green I to a fluorescence intensity ratio or change of silver nanoclusters based on a concentration of a drug (Gemcitabine) injected to cells producing ATP.

Referring to FIG. 9A, it can be seen that as the concentration of a drug (Gemcitabine) that can kill ATP-producing cells increases, the concentration of ATP produced in the cells decreases. As the concentration of ATP decreases, the fluorescence intensity change or ratio of SYBR Green I is increased. The fluorescence intensity change or ratio of SYBR Green I was measured as the ratio of the fluorescence intensity of SYBR Green I prior to the injection of the drug (Gemcitabine) relative to the fluorescence intensity of SYBR Green I after the injection based on the concentration of the injected drug (Gemcitabine).

Referring to FIG. 9B, it can be seen that as the concentration of the drug (Gemcitabine) that can kill ATP-producing cells increases, the concentration of ATP decreases. As the concentration of ATP decreases, the fluorescence intensity ratio or change of silver nanocluster decreases. The fluorescence intensity ratio or change of the silver nanocluster was measured as the ratio of the fluorescence intensity of the silver nanocluster prior to the injection of the drug (Gemcitabine) relative to the fluorescence intensity of the silver nanocluster after the injection based on the concentration of the injected drug (Gemcitabine).

Referring to FIG. 9C, the ratio () of the fluorescence intensity ratio or change of SYBR Green I to the fluorescence intensity ratio or change of the silver nanocluster measured based on the concentration of drug Gemcitabine is close to 1. The change amount of fluorescence intensity of SYBR Green I corresponds to the change amount of fluorescence intensity of the Ag nanoclusters.

This indicates that as for the present biochemical molecule detection sensor, the biochemical molecule is normally bound to the complexes and the detection error is low, and, thus, the detection accuracy of the biochemical molecule is high.

Moreover, in one embodiment, the operations of measuring the fluorescence intensities of the first and second fluorescence and/or determining absence/presence of the first and second fluorescence may be realized by imaging with a camera. The imaging by the camera is also possible using a conventional smartphone camera. It is also possible to take an image using a smartphone camera and to quantify data based on the taken image.

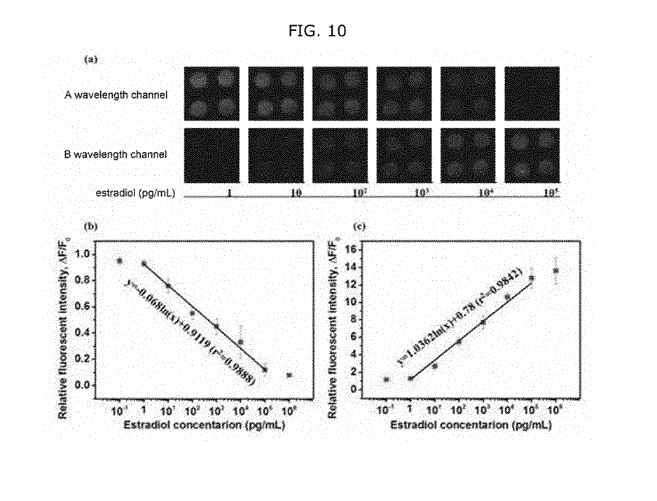

FIG. 10 shows quantification data based on concentrations of a target biomolecule (estradiol) for two fluorescence wavelength bands (A and B), using a biochemical molecule detection sensor according to the present disclosure. In FIG. 10, the image is imaged using a smartphone camera and the data is quantified based on the taken image.

The smartphone camera may be used a camera of a Samsung Galaxy S4 smartphone. The first aptamer complex contains an estradiol DNA aptamer A sequence tagged with an amine functional group, a complementary DNA to the estradiol DNA aptamer A sequence, and SYBR Green I. The second aptamer complex contains an estradiol DNA aptamer B sequence tagged with an amine functional group, and 50 nm carboxylate YO fluorescent polystyrene bead. Further, the substrate is embodied as a slide glass with Al₂O₃ (30 nm)/Ag (120 nm) deposited thereon. An OH functional group is formed on the Al₂O₃ surface of the substrate, which, in turn, is subjected to APTES treatment to produce an amine functional group thereon. Further, the substrate is subjected to glutaraldehyde treatment, resulting in aldehyde functional group formed thereon. Then, the first aptamer complex is microspotted (2×2, diameter 100 micron) onto the substrate through an ink jet printer.

A following table 2 indicates comparison of the detection accuracy values between using the portable smartphone camera and using ELISA. As shown in Table 2, upon applying the present disclosure, it is possible to obtain detection accuracies using the smartphone comparable to those using ELISA though they are not superior to those using the ELISA.

TABLE 2

| | | | Accuracy | |
|---|---|---|---|---|
| Detection | | | (Area Under | Standard |
| method | Sensitivity % | Specificity % | ROC Curve) | Deviation |
| ELISA | 88.9 | 100 | 0.956 | 0.048 |
| Double | 77.8 | 80 | 0.922 | 0.064 |
| quantification | | | | |
| with camera | | | | |

The above description is not to be taken in a limiting sense, but is made merely for the purpose of describing the general principles of exemplary embodiments, and many additional embodiments of this disclosure are possible. It is understood that no limitation of the scope of the disclosure is thereby intended. The scope of the disclosure should be determined with reference to the Claims.

What is claimed is:

1. A biochemical molecule detection sensor comprising:
a substrate;
a first aptamer complex chemically bound to the substrate, wherein the first aptamer complex selectively binds to a biochemical molecule, and includes at least one double helix DNA into which a fluorescent dye generating first fluorescence with a first wavelength is intercalated; and
at least one second aptamer complex selectively binding to the biochemical molecule and generating second fluorescence with a second wavelength, wherein the first wavelength is different from the second wavelength,
wherein fluorescence intensities of the first and second fluorescence depend on an amount of the biochemical molecule reacting with the first aptamer complex and the second aptamer complex.

2. The sensor of claim 1, wherein the double helix DNA includes:
a first nucleic acid strand having a first nucleotide sequence selectively binding to the biochemical molecule; and
a second nucleic acid strand having a second nucleotide sequence complementary to the first nucleotide sequence, wherein the second nucleic acid strand is complementary to the first nucleic acid strand,
wherein the fluorescent dye is intercalated into between the first nucleic acid strand and the second nucleic acid strand.

3. The sensor of claim 1, wherein the fluorescent dye includes at least one selected from a group consisting of SYBR Green I, thiazole orange, and ethidium bromide.

4. The sensor of claim 2, wherein the second aptamer complex includes:
a third nucleic acid strand having the first nucleotide sequence selectively binding to the biochemical molecule; and
a phosphor coupled to the third nucleic acid strand, wherein the phosphor generates the second fluorescence.

5. The sensor of claim 2, wherein the first nucleic acid strand is labeled with a functional group chemically bound to the substrate, wherein the functional group includes at least one selected from a group consisting of amine group, carboxyl group, maleimide group or thiol group.

6. The sensor of claim 4, wherein the phosphor includes at least one selected from a group consisting of quantum dot, fluorescent dye and metal nanocluster.

7. A method for detecting a biochemical molecule using multi-wavelength fluorescence, the method comprising:
applying a reagent onto a first aptamer complex, wherein the first aptamer complex is chemically bound to the substrate, wherein the first aptamer complex selectively binds to the biochemical molecule, and includes at least one double helix DNA into which a fluorescent dye generating first fluorescence with a first wavelength is intercalated, wherein the reagent includes the biochemical molecule and at least one second aptamer complex selectively binding to the biochemical molecule and generating second fluorescence with a second wavelength, wherein the first wavelength is different from the second wavelength;
removing the second aptamer complex not bound to the biochemical molecule; and
measuring fluorescence intensities of the first and second fluorescence.

8. The method of claim 7, wherein the double helix DNA includes:
a first nucleic acid strand having a first nucleotide sequence selectively binding to the biochemical molecule; and
a second nucleic acid strand having a second nucleotide sequence complementary to the first nucleotide sequence, wherein the second nucleic acid strand is complementary to the first nucleic acid strand,
wherein the fluorescent dye is intercalated into between the first nucleic acid strand and the second nucleic acid strand.

9. The method of claim 8, wherein the second aptamer complex includes:
a third nucleic acid strand having the first nucleotide sequence selectively binding to the biochemical molecule; and
a phosphor coupled to the third nucleic acid strand, wherein the phosphor generates the second fluorescence.

10. The method of claim 7, further comprising:
repeating the operations (a) to (c) using varying concentrations of the biochemical molecule; and
deriving an analysis function for fluorescence intensities of the first and second fluorescence based on the concentration of the biochemical molecule.

11. The method of claim 7, further comprising: detecting the biochemical molecule by determining whether the intensity of the first fluorescence increases or decreases and whether the second fluorescence occurs or not.

12. The method of claim 7, further comprising: detecting the biochemical molecule by determining whether the intensity of the first fluorescence increases or decreases and whether the intensity of the second fluorescence increases or decreases.

13. The method of claim 7, wherein the operation (c) is carried out by imaging using a camera.
